(19) United States
(12) Patent Application Publication
(10) Pub. No.: US 2005/0137242 A1
(43) Pub. Date: Jun. 23, 2005

(54) Betamimetics having a long-lasting activity, processes for preparing them, and their use as medicaments

(75) Inventors: Alexander Walland, Ingelheim (DE); Kurt Schromm, Ingelheim (DE); Karl-Heinz Bozung, Mainz (DE); Herman Schollenberger, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 11/053,514

(22) Filed: Feb. 8, 2005

Related U.S. Application Data
(63) Continuation of application No. 09/836,462, filed on Apr. 18, 2001, now abandoned.

(30) Foreign Application Priority Data
Apr. 27, 2000 (EC) SP00-3424
Oct. 17, 2000 (DE) 100 51 318.2

(51) Int. Cl. A61K 31/4184; C07D 235/24

(52) U.S. Cl. 514/394; 548/309.7

(57) ABSTRACT

A compound of formula 1 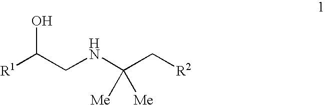 wherein:
R¹ is a group 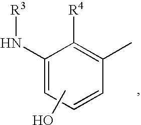
   wherein R³ is a benzyl group optionally substituted by a methoxy group; R⁴ is a hydrogen atom, or R³ and R⁴ together are a —CO—CH₂—O— bridge, the carbonyl group of the bridge being bound to the nitrogen; and
R² is a group selected from 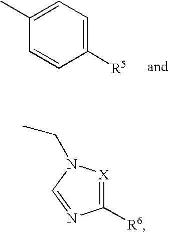 wherein
   R⁵ is a dimethylamino, methoxy, or butoxy group, X is a nitrogen or a carbon atom, and
   R⁶ is a methoxyphenyl group, if X is nitrogen, or is an anellated phenyl ring also linked to X, if X is carbon, or the individual optical isomers, mixtures of the individual enantiomers, racemates, or acid addition salt thereof.

### RELATED APPLICATIONS

This application is a continuation of U.S. Ser. No. 09/836,462, filed Apr. 18, 2001, now abandoned, which claimed priority to German Application No. 100 51 318.2, filed Oct. 17, 2000, and Ecuadorian Application No. SP00-3424, each of which is hereby incorporated by reference in its entirety.

### FIELD OF THE INVENTION

The present invention relates to new betamimetics of formula 1 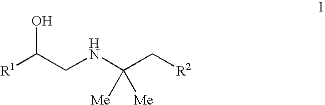
wherein the groups R¹ and R² have the meanings given in the claims and specification, processes for preparing them, and their use as medicaments.

### BACKGROUND OF THE INVENTION

Betamimetics (β-adrenergic substances) are known from the prior art. They may be used in a variety of therapeutic applications.

For drug treatment of diseases, it is often desirable to prepare medicaments with a longer duration of activity. As a rule, this ensures that the concentration of the active substance in the body needed to achieve the therapeutic effect is present over a longer period of time without the need to administer the drug repeatedly and frequently. The administration of an active substance at longer intervals of time also contributes considerably to the patient's well-being.

The aim of the present invention is to prepare betamimetics which are characterized by a longer duration of activity and can thus be used to prepare pharmaceutical compositions which have a longer-lasting activity.

### DETAILED DESCRIPTION OF THE INVENTION

Surprisingly, it has been found that the aim specified above is solved by compounds of formula 1.

Accordingly the present invention relates to compounds of formula 1 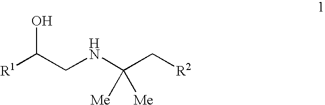
wherein:
R¹ is a group 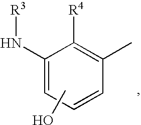
   wherein
      R³ is benzyl group optionally substituted by a methoxy group,
      R⁴ is a hydrogen atom, or
      R³ and R⁴ together are a —CO—CH₂—O— bridge, the carbonyl group of this bridge being bound to the nitrogen; and
R² is a group selected from 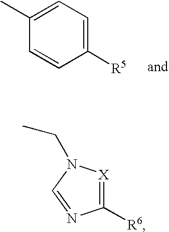
   wherein
      R⁵ is a dimethylamino, methoxy, or butoxy group,
      X is a nitrogen or a carbon atom, and
      R⁶ is a methoxyphenyl group, if X is nitrogen, or is an anellated phenyl ring, which is also linked to X, if X is carbon.

Preferred compounds of formula 1 are those wherein
R¹ is a group selected from 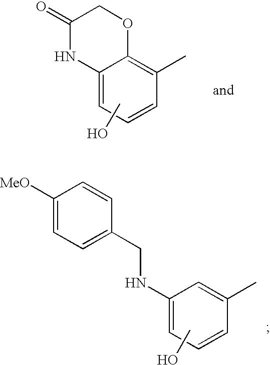
   and
R² is a group selected from

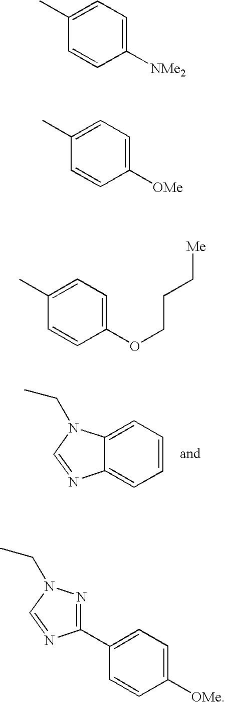

Particularly preferred are compounds of formula 1, wherein:
R¹ is a group selected from 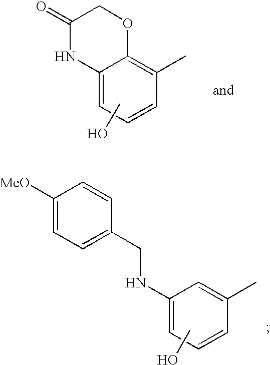
   and
R² is a group selected from

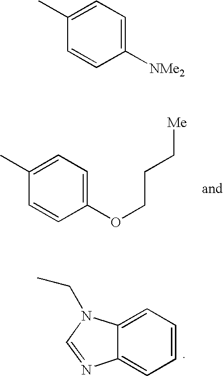

Of particular importance according to the invention are compounds of formula 1, wherein
R¹ is a group 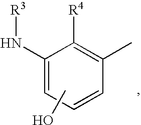
   wherein R³ and R⁴ together are a —CO—CH₂—O— bridge, the carbonyl group of this bridge being bound to the nitrogen; and
R² is a group selected from 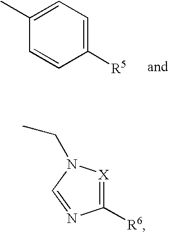
   wherein
      R⁵ is a dimethylamino, methoxy, or butoxy group,
      X is a nitrogen or a carbon atom, and
      R⁶ is a methoxyphenyl group, if X is nitrogen or an anellated phenyl ring which is also linked to X, if X is carbon.

Preferred compounds of formula 1 are those wherein
R¹ is 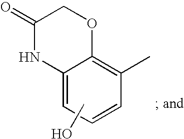
R² is a group selected from

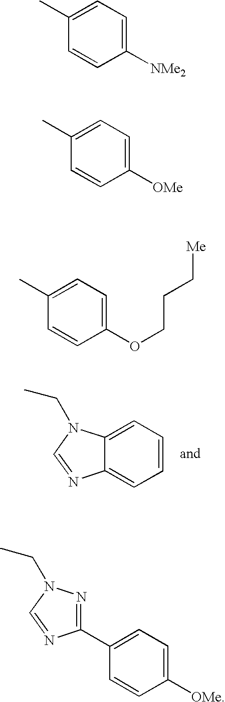

Of equivalent importance according to the invention are compounds of formula 1, wherein
R¹ is a group 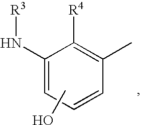
   wherein
      R³ is a benzyl group optionally substituted by a methoxy group, and
      R⁴ is a hydrogen atom; and
R² is a group 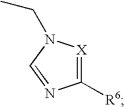
   wherein
      X is a nitrogen or a carbon atom, and
      R⁶ is a methoxyphenyl group, if X is nitrogen, or an anellated phenyl ring which is also linked to X, if X is carbon.

Of outstanding importance according to the invention are the following compounds of formula 1:
a. 1-[3-(4-methoxybenzylamino)-4-hydroxyphenyl]-2-[4-(1-benzimidazolyl)-2-methyl-2-butylamino]ethanol;
b. 1-[2H-5-hydroxy-3-oxo-4H-1,4-benzoxazin-8-yl]-2-[3-(4-N,N-dimethylaminophenyl)-2-methyl-2-propylamino]ethanol; and
c. 1-[2H-5-hydroxy-3-oxo-4H-1,4-benzoxazin-8-yl]-2-[3-(4-n-butyloxyphenyl)-2-methyl-2-propylamino]ethanol.

In the compounds of formula 1 according to the invention, R¹ may be the group 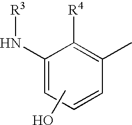
and preferably one of the groups

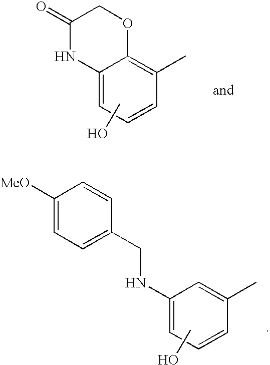

Of the compounds of formula 1 according to the invention, the ones which are particularly preferred are those wherein the hydroxyl group in the abovementioned groups R¹ is in the ortho or meta position relative to the amino substituent. Most preferably, the hydroxy group is in the ortho position to the amino group.

The invention relates to the compounds of formula 1 optionally in the form of the individual optical isomers, mixtures of the individual enantiomers or racemates as well as in the form of the free bases or the corresponding acid addition salts thereof with pharmacologically acceptable acids, such as, for example, acid addition salts with hydrohalic acids (e.g., hydrochloric or hydrobromic acid) or organic acids such as acetic, oxalic, fumaric, diglycolic, or methanesulfonic acid. Of the acid addition salts mentioned above, the salts of hydrochloric, methanesulfonic, and acetic acid are particularly preferred according to the invention.

The compounds according to the invention may be prepared, as described below, partly analogously to procedures which are already known in the prior art (Scheme 1 below). 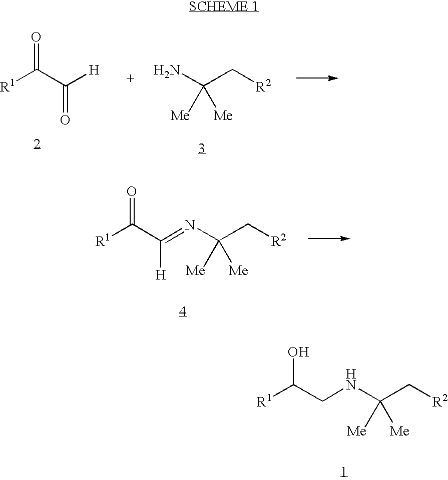
Scheme 1

Starting from suitably substituted aldehydes 2, which may optionally be present in the form of their hydrates, the reaction is carried out with the amines 3 to form the Schiff's bases of formula 4. Methods of forming Schiff's bases are known from the prior art. These Schiff's bases are finally reduced to form the compounds of formula 1 according to the invention. This reduction may be carried out, for example, with metal salt hydrides of the sodium borohydride type analogously to known standard methods. It may possibly be necessary to use protecting groups (e.g., a benzyl protecting group); their use and subsequent removal are known to those skilled in the art.

The Examples of synthesis described below serve to illustrate the present invention further. They must only be taken as examples of procedure, to illustrate the invention further, without restricting the invention to the object described below by way of example.

### EXAMPLE 1

### 1-[2H-5-hydroxy-3-oxo-4H-1,4-benzoxazin-8-yl]-2-[3-(4-N,N-dimethylaminophenyl)-2-methyl-2-propylamino]ethanol

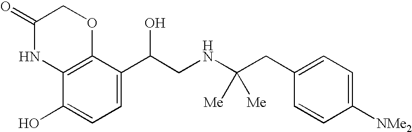

### Preparation of the Schiff's Base (Compound of Formula 4)

19.1 g (0.058 mol) of [2H-5-benzyloxy-3-oxo-4H-1,4-benzoxazin-8-yl]glyoxal hydrate is added to a solution of 250 mL of ethanol and 9.6 g (0.05 mol) of 3-(4-N,N-dimethylaminophenyl)-2-methyl-2-propylamine heated to 70° C. and stirred for 15 minutes. After cooling, the crystals precipitated are suction filtered and dried.

Yield: 24 g (99% of theory); melting point: 201° C.-204° C.

### Reduction of the Schiff's Base to obtain 1-[2H-5-benzyloxy-3-oxo-4H-1,4-benzoxazin-8-yl]-2-[3-(4-N,N-dimethylaminophenyl)-2-methyl-2-proplamino]ethanol

24 g of the Schiff's base (0.0495 mol) obtained is suspended in a mixture of 120 mL of ethanol/120 mL of dioxane and combined with 2 g of NaBH₄ within 30 minutes at 10° C.-20° C. and stirred for one hour. After the addition of 10 mL of acetone, the mixture is stirred for 30 minutes, diluted with 300 mL of ethyl acetate, the ethyl acetate phase is washed twice with about 200 mL of water, dried with sodium sulfate, and the solvent is distilled off in vacuo. The dihydrochloride is isolated from the residue with alcohol/acetone by acidifying with concentrated hydrochloric acid and suction filtering.

Yield: 17.5 g (62.6% of theory); melting point: 180° C.-185° C.

### Cleaving of the Protecting Group to Obtain the Title Compound

3.5 g of the benzyl compound obtained above (0.0066 mol) is hydrogenated in 75 mL of methanol with the addition of 0.5 g of Pd/C at ambient temperature and normal pressure. The catalyst is suction filtered, the filtrate is evaporated down, screened, and the crystals precipitated are separated off.

Yield: 2.4 g (82.8% of theory); melting point: 216° C.-218° C. (hydrochloride).

### EXAMPLE 2

### 1-[2H-5-hydroxy-3-oxo-4H-1,4-benzoxazin-8-yl]-2-[3-(4-n-butyloxyphenyl)-2-methyl-2-propylamino]ethanol

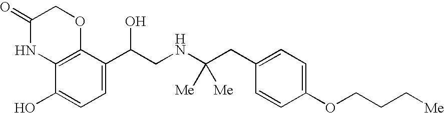

The title compound is prepared analogously to the method in Example 1.

Melting point: 189° C.-190° C. (methanesulfonate).

### EXAMPLE 3

### 1-[3-(4-methoxybenzylamino)-4-hydroxyphenyl]-2-[4-(1-benzimidazolyl)-2-methyl-2-butylamino]ethanol

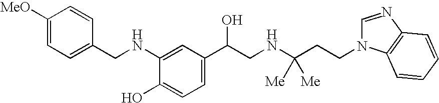

The title compound is prepared analogously to the method in Example 1.

Melting point: 154° C.-155° C. (acetate).

### EXAMPLE 4

### 1-[2H-5-hydroxy-3-oxo-4H-1,4-benzoxazin-8-yl]-2-[3-(4-methoxyphenyl)-2-methyl-2-propylamino]ethanol

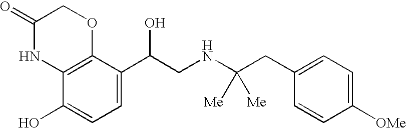

The title compound is prepared analogously to the method in Example 1.

Melting point: 202° C.-205° C. (hydrochloride).

### EXAMPLE 5

### 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol

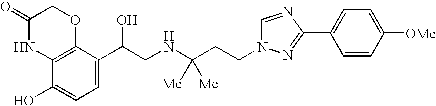

The title compound is prepared analogously to the method in Example 1.

Melting point: 175° C.-179° C. (hydrochloride).

As has been found, the compounds of formula 1 are characterized by their range of uses in the therapeutic field. Particular mention should be made of those applications for which the compounds of formula 1 according to the invention may preferably be used on the basis of their pharmaceutical activity as betamimetics. These include, for example, the treatment of bronchial asthma (relaxation of the bronchial muscle), the treatment of the inflammatory component in COPD, the inhibition of premature labor in midwifery (tocolysis), the restoration of the sinus rhythm in the heart in cases of atrio-ventricular block as well as the correcting of bradycardiac heart rhythm disorders (antiarrhythmic agent), the treatment of circulatory shock (vasodilatation and increasing the heart-time volume) as well as the treatment of itching and skin inflammation.

The compounds of formula 1 may be used on their own or in conjunction with other active substances of formula 1 according to the invention. If desired, the compounds of formula 1 may also be used in conjunction with other pharmacologically active substances. These may be, in particular, anticholinergics, possibly other betamimetics, antiallergics, PAF antagonists, leukotriene antagonists, and steroids, as well as combinations of active substances.

Examples of anticholinergics which may be mentioned include ipratropium bromide, oxitropium bromide, and particularly tiotropium bromide. Drug combinations which contain tiotropium bromide as an additional active substance as well as the compounds of formula 1 according to the invention are particularly preferred according to the invention.

This combination is particularly important in the treatment of asthma or COPD, particularly COPD.

Suitable preparations for administering the compounds of formula 1 include, for example, tablets, capsules, suppositories, solutions, etc. The content of the pharmaceutically active compound(s) should be in the range from 0.05 to 90 wt. %, preferably 0.1 to 50 wt. %, of the composition as a whole. Suitable tablets may be obtained, for example, by mixing the active substance(s) with known excipients, for example, inert diluents such as calcium carbonate, calcium phosphate, or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example, collidone or shellac, gum arabic, talc, titanium dioxide or sugar. To achieve delayed release or prevent incompatibilities, the core may also consist of a number of layers. Similarly, the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol, or sugar and a flavor enhancer, e.g., a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g., with the addition of isotonic agents, preservatives such as p-hydroxybenzoates, or stabilizers such as alkali metal salts of ethylenediamine tetraacetic acid (EDTA), optionally using emulsifiers and/or dispersants, whereas if water is used as the diluent, for example, organic solvents may optionally be used as solvating agents or dissolving aids, and transferred into injection vials or ampoules or infusion bottles.

Capsules containing one or more active substances or combinations of active substances may, for example, be prepared by mixing the active substances with inert carriers such as lactose or sorbitol and packing them into gelatine capsules. Suitable suppositories may be made, for example, by mixing with carriers provided for this purpose, such as neutral fats or polyethyleneglycol or the derivatives thereof. Excipients which may be used include, for example, water, pharmaceutically acceptable organic solvents such as paraffins (e.g., petroleum fractions), vegetable oils (e.g., groundnut or sesame oil), mono- or polyfunctional alcohols (e.g., ethanol or glycerol), carriers such as natural mineral powders (e.g., kaolins, clays, talc, chalk), synthetic mineral powders (e.g., highly dispersed silicic acid and silicates), sugars (e.g., cane sugar, lactose, and glucose), emulsifiers (e.g., lignin, spent sulfite liquors, methylcellulose, starch, and polyvinylpyrrolidone) and lubricants (e.g., magnesium stearate, talc, stearic acid, and sodium lauryl sulfate).

The preparations are administered by the usual methods, preferably by inhalation in the treatment of asthma or COPD. For oral administration, the tablets may, of course, contain, apart from the abovementioned carriers, additives such as sodium citrate, calcium carbonate, and dicalcium phosphate together with various additives such as starch, preferably potato starch, gelatine, and the like. Moreover, lubricants such as magnesium stearate, sodium lauryl sulfate, and talc may be used at the same time for the tabletting process. In the case of aqueous suspensions, the active substances may be combined with various flavor enhancers or colorings in addition to the excipients mentioned above.

The dosage of the compounds according to the invention is naturally highly dependent on the method of administration and the complaint which is being treated. When administered by inhalation, the compounds of formula 1 are characterized by a high potency even at doses in the μg range. The compounds of formula 1 may also be used effectively above the μg range. The dosage may then be in the gram range, for example.

The examples of formulations which follow illustrate the present invention without restricting its scope:

Examples of Pharmaceutical Formulations

| A. Tablets | per tablet |
|---|---|
| active substance | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose, and some of the corn starch are mixed together. The mixture is screened, then moistened with a solution of polyvinylpyrrolidone in water, kneaded, wet-granulated, and dried. The granules, the remaining corn starch, and the magnesium stearate are screened and mixed together. The mixture is compressed to produce tablets of suitable shape and size.

| B. Tablets | per tablet |
|---|---|
| active substance | 80 mg |
| lactose | 55 mg |
| corn starch | 190 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened.

The sodium carboxymethyl starch and the magnesium stearate are added and mixed in and the mixture is compressed to form tablets of a suitable size.

| C. Ampoule solution | | |
|---|---|---|
| active substance | 50 | mg |
| sodium chloride | 50 | mg |
| water for inj. | 5 | mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilized and sealed by fusion. The ampoules contain 5 mg, 25 mg, and 50 mg of active substance.

| D. Metering aerosol | |
|---|---|
| active substance | 0.005 |
| sorbitan trioleate | 0.1 |
| monofluorotrichloromethane and difluorodichloromethane (2:3) | ad 100 |

The suspension is transferred into a conventional aerosol container with a metering valve. Preferably, 50 μl of suspension are delivered per spray. The active substance can also be in a higher dose if desired (e.g., 0.02 wt. %).

| E. Solutions (in mg/100 mL) | |
|---|---|
| active substance | 333.3 mg |
| tiotropium bromide | 333.3 mg |
| benzalkonium chloride | 10.0 mg |
| EDTA | 50.0 mg |
| HCl (1 N) | ad pH 3.4 |

This solution can be produced in the usual way.

| F. Inhalable powder | | |
|---|---|---|
| active substance | 6 | μg |
| tiotropium bromide | 6 | μg |
| lactose monohydrate | ad 25 | mg |

The inhalable powder is prepared in the usual way by mixing the individual ingredients together.

1. A compound of formula 1 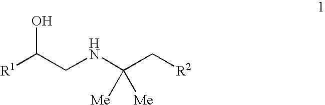
wherein:
R¹ is 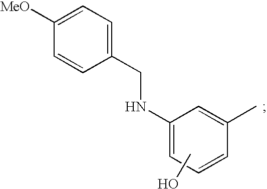
and
R² is 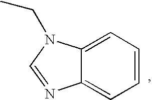
or an acid addition salt thereof.

2. The compound of formula 1 according to claim 1, wherein the hydroxy group in the group R¹ is in the ortho or meta position to the amino group.

3. 1-[3-(4-methoxybenzylamino)-4-hydroxyphenyl]-2-[4-(1-benzimidazolyl)-2-methyl-2-butylamino]ethanol, or an acid addition salt thereof.

4. The compound according to one of claims 1, 2, or 3, wherein the acid addition salt thereof is formed with a pharmacologically acceptable acid.

5. A method of treating bronchial asthma, the inflammatory component in COPD, premature onset of labor in midwifery (tocolysis), atrio-ventricular block, bradycardiac hearth rhythm disorders, circulatory shock, or itching and inflammation of the skin in a host in need of such treatment, the method comprising administering to the host the compound according to one of claims 1, 2, or 3.

6. A pharmaceutical composition comprising a compound according to one of claims 1, 2, or 3 and a conventional excipient or carrier.

7. The pharmaceutical composition according to claim 6, further comprising at least one other active substance selected from the group consisting of anticholinergics, betamimetics, antiallergics, PAF antagonists, leukotriene antagonists, and steroids.

8. The pharmaceutical preparation according to claim 7, further comprising tiotropium bromide.
